(19) United States
(12) Patent Application Publication
(10) Pub. No.: US 2018/0117105 A1
(43) Pub. Date: May 3, 2018

(54) Indian wax water/ zea mays

(71) Applicant: Katrina M. Lundy, Wai Pahu, HI (US)

(72) Inventor: Katrina M. Lundy, Wai Pahu, HI (US)

(21) Appl. No.: 15/330,671

(22) Filed: Oct. 27, 2016

(51) Int. Cl. A61K 36/02; A61K 35/644; A61K 9/00

(52) U.S. Cl. CPC A61K 36/02; A61K 9/0095; A61K 35/644

(57) ABSTRACT

Indian Wax Water/Indian Algae is a distinct new water technology using different kinds of waxes and plant extracts to create, promote health—healing, medicine, medicinal mixtures, beauty, purification methods, cell regeneration, organic matter, gardening, hydroponics, new plant forms, Indian Algae, futuristic products based on wax water technology. New molecule structures and polymerization promotes healing for HIV, AIDS and other numerous diseases, E.coli, parasitic disorders internal or external. In combination with natural water types—spring, rain, sulfate, ocean and catalyst, mineral enhanced, purified, distilled, carbonated, and botanical.

### TECHNICAL FIELD

This invention relates to water technology and methods applied for producing organic purified water that enhances food vitamin-mineral content growing super foods, new plant forms, Indian Algae and many other uses including medicine, ointments, perfumes, health and beauty products.

### BACKGROUND OF THE INVENTION

Indian Wax Water/Indian Algae is created from studies into ancient Indian cultures congruous with the Mayans, Aztecs, Incas, Native American Indians, Hawaiians, Polynesians, Indians of India and other tribal peoples of ethnic Indian nationalities.

Applying herbal knowledge of trees, plant parts, agriculture and cultivation methods, rain catchers, protein distillates, plants, such as dog grass, juniper, eupatorium purpuream, rubber plants, balms, essential oils, salts, tinctures and so forth that were beneficial to ancient Indians and utilized by old time medicine men, high priest, kahunas, wise men and shaman. Indian Algae is an idea or ideas taken from the old ways and mixed with new concepts of the healing arts—20^{th} century stuff, materials based on cell regeneration, hydroponic water gardens, enzyme compatibility and anti-enzyme, healing salts, trace minerals, plasticizers, glycosphingolipids, new age physics, physiology, neurochemistry, petroleum distillates, waxes, and natural colors.

For instance, the wild blue green algae Aphanizomenon Flos-aquae and a number of blue green or algae species are known to have been cultivated and used by the Mayan, Aztec, and Incan Indians. They are all similar to other fresh water algaes but not able to flourish in society today regarding water pollution such as lead methyl, mercury, pesticides, environmental toxins and contaminates in the soil and fresh water supply. In reference to The Regeneration Effect by Dr. John W. Apsely, pg. 52 and chapter 2. Indian Wax Water/Indian Algae is a preferably more valuable type of fresh water algae of exceptionally high quality, pertaining to methods and techniques of cultivation. The water is controlled allowing for plant growth and a purification process to take place, after just two weeks into the growth cycle, the Indian water can be harvested and ready for a mineral drink. I am concluding in my travels to Central America, Mexico, Yucatan, San Salvador, Guatemala, Honduras, Polynesia, U.S., and Canada that water methods of purification such as Indian Wax Water can be successfully stored and used in third world countries.

In reference to Molecular Biology of the Cell Fourth Edition 2002, pg. 350 proteins p. 355, 362, 380, 382, DNA by Bruce Alberts, Julian Lewis, Keith Roberts, Peter Walter, Alexander Johnson, Martin Raff.

Encyclopedia of Genetics 2004 Vol. 1, Hybridization and Introgression, Jeffrey A. Knight, Ph. D. and Vol. 2, Hybridomas and Monoclonal Antibodies-XYY Syndrome pg. 174, 175, 208, 209, 35, 36.

Disease Related, Prevention—Vol. 2 pg. 729, telomerase enzymes.

Honolulu Star Advertiser—Disease Possibly Raises HIV Risk, Sun. 5-11-2014 pg. A7 AIDS.

### BRIEF SUMMARY OF THE INVENTION

Indian Wax Water/Zea Mays, Indian Algae, will contribute to planet earth in solving world-wide water problems, new ideas, concepts in water purification methods. The advantages including new water systems without using harmful chemicals, eliminating pestilence problems, new ways of growing plants, developing hybrids and superfoods; also, water ponds or swimming pools that are clean enough that an animal may drink from and perhaps grow plants and fish at the same time. Thereby creating and establishing water fountains and bird baths that keep animals clean and are environmentally safe for public places such as parks and recreation areas. As we evolve into the future we may see many uses for Indian Wax Water, mega medicinal benefits, health and beauty products with anti-aging properties, protein hair conditioners that reconstruct healthy hair, feel good nutritional drinks that really work, skin protective creams and much more of the several multi-various uses for Indian Wax Water. In regarding most traditional furfural mixtures that are made with corn or corn syrup, they are not to be mixed with wax, exceptions of 0.01 that could result in chloroform, besides they are ethanol mixtures and preserve fine alone.

### BRIEF DESCRIPTION OF THE DRAWINGS

A drawing taken from a flier shows an exact recipe in the process of making Indian Wax Water, 100% organic and can be made in plastic or glass containers as shown in:

FIG. 1 illustrates a method of preparing Indian Wax Water according to a recipe, step 1 and 2. Water is obtained from a natural source, rain, natural spring, well water, pure and simple. Approximately ½ teaspoon of natural bee wax is then added to 1 gallon of the water and set aside for two weeks to a month. After one month water is ready to be mixed and flavored for an enzyme drink, following step 2 in a clay mug add 2 cups of Indian wax Water with 1 and ½ tablespoons honey and 2 drips spearmint essential oil or peppermint, then stir and drink. This particular recipe is enzyme compatible and a 100% organic also complementing other enzyme food combinations.

Also shown in art FIG. 1, FIG. 2A, FIG. 2B, FIG. 3 new plant forms growing in the Indian Wax Water developing into geometrical rectangular shapes and patterns. The color varies somewhat depending on the amounts of sunlight and other factors; it appears to be forest green.

FIG. 2A shows a synchronized view of the plant form Indian Algae clusters in art color drawing, forest green.

FIG. 2B shows a synchronized view of the plant form Indian Algae clusters in a black and white print.

FIG. 3 shows a synchronized view of the plant form Indian Algae; clusters in art color drawing, forest green.

This method of hydroponic gardening of growing Indian Algae is highly beneficial and can be applied or used in repairing other types of water plants, algae perhaps the blue green species, sea asparagus and many more.

### DETAILED DESCRIPTION OF THE INVENTION

### The Best Mode for Carrying Out Invention

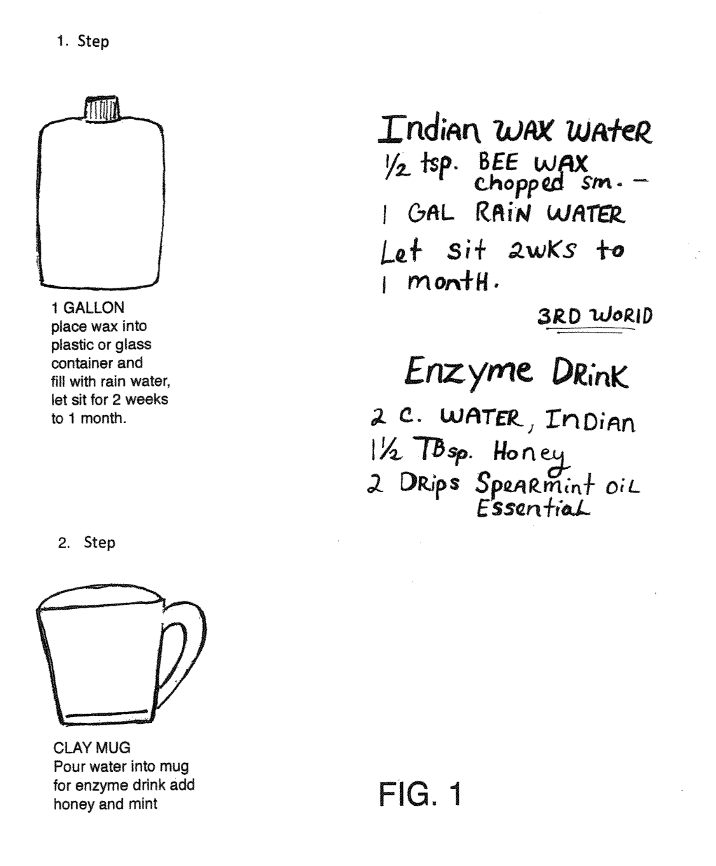

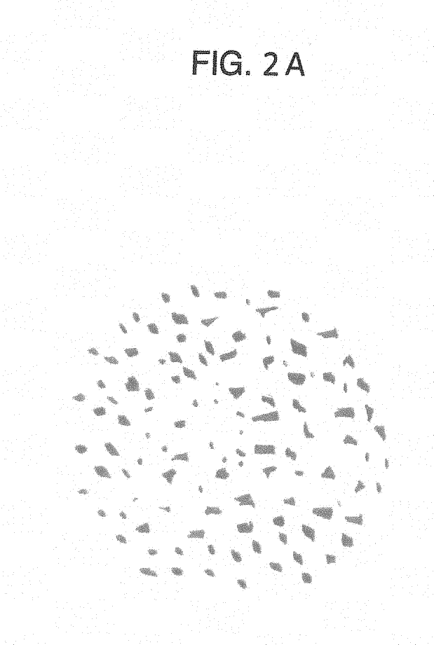

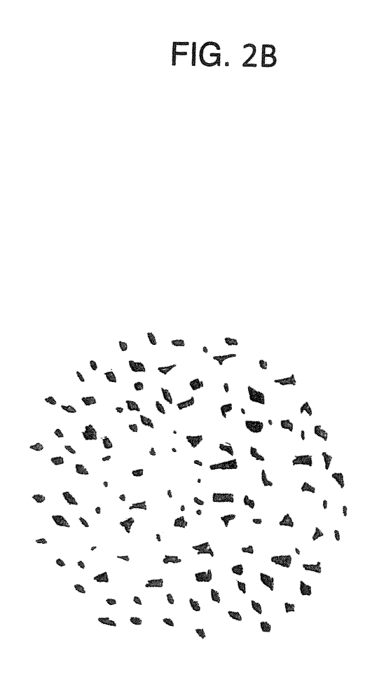

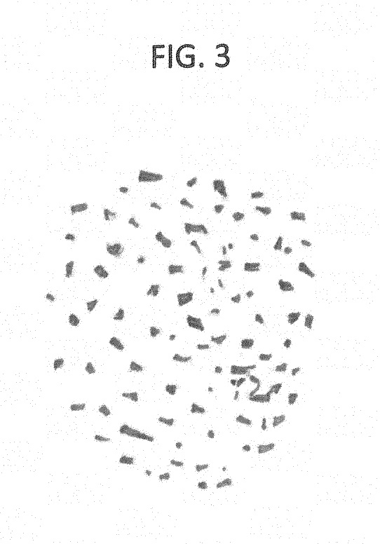

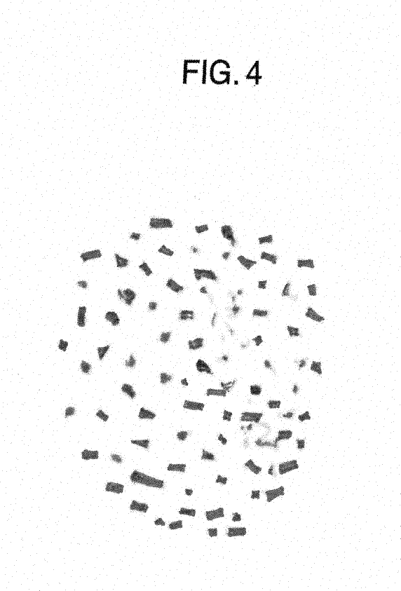

The process of making and using Indian Wax Water, Indian Algae is a hydroponic process involving multi-varieties of waxes in combination with specific water types to create new plant forms, medicine, health and beauty products. It is similar to blue green algae, grown in fresh water lakes but not identical. Indian Algae shown in FIG. 1, FIG. 2A, FIG. 2B and FIG. 3 which produces a vegetable derived protein imitating cellular membrane complex, can be used as a flavor enhancer or in health food drinks—directly repairing DNA and needful for body builders, also a nutritional drink with super food power. Deoxyribonucleic acid, the complex substance that makes up genes, it contains the genetic information for all organisms. In particular the organic bees wax included in the Indian Wax Water is enzyme compatible and simply divine when used for gardening purposes, supplying plants with enriched vitamins and minerals producing lush juicy fruits and vegetables. The bee wax cleans and purifies the water removing parasites and unwanted pests by a fermentation process. Also Indian Algae/Wax Water is excellent for skin, hair and beauty product emollients, creams lotions and cosmetics that feed nutrients into the skin, repairing wrinkles with its sulfur content and enabling cell regeneration to take place. Like most catalyst waters it binds enzymes and molecules promoting healing for many skin problems, And also like most algae that contain natural amounts of vitamin A, help to generate healing for the skin, assisting in the furtherance of cell renewal. Unlike types of synthetic vitamin A, or non-vitamin A which probably do more damage than good—in certain cases it was found to contain Cancer causing properties, the individual developed an aplastic anemia and died.

Indian Algae is also excellent when added into medicated make-up that both covers and treats the skin. When applied as a astringent with limewater the end product showed promising results. Medicinally it can be used to help treat most Cancers caused by cell mutations or others including cancers of the skin, by also cleansing in an Indian bath of wax water and castile soap, properly washing out toxins, contaminates, lead and other harmful food additives. Excreting pollutants through the pores of the skin, sweat and urine thus allowing the body to repair and preserve cells. Also it can be used as a blood cleanser for bone marrow, HIV AIDS, glandular, lymph diseases, obesity, diabetes with specific water types, emissions generated by electronic devices, radiation and of course lead water poisoning, arthritis, depression, the many neurological disorders and exceedingly so much more as we can always learn from our past with ancient Indian remedies and how they used algae as a flavor enhancer. Perhaps just a table spoon sprinkled over food as a condiment or beverage that provided an excellent food supplement giving them strength, good health, peace of mind, zest and vitality.

I claim the invention of indian wax water/

zea mays

indian algae:

1. Is a new water technology, a process claim

2. Forms a new plant species

3. Used in hydroponic, organic gardening, grows super foods and increases vitamins and minerals in plant growth production.

4. Multi medicinal value, restores health in general.

5. Promotes healing for HIV AIDS.

6. Promotes healing for E. coli.

7. Promotes healing for Ebola virus in combination with specific type waxes.

8. Promotes healing for eczema, skin disorders, cancers beneath the skin, used externally with botanicals or in protective creams.

9. Promotes healing for cancers, an organic inhibitor to prevent growth of cells.

10. Promotes healing as a blood cleanser for parasitic disorders, expels worms.

11. Improves vision, helps eye disorders.

12. Used as a mouthwash, promotes healing for gum diseases.

13. Creates cell regeneration.

14. Repairs DNA, nitrogen nucleobases, interaction between DNA and other proteins, amino acids.

15. Creates new enzyme molecule structures.

16. Creates polymerization used used with cosmetics in conjunction with cellulose gums, resistant to bacterial decomposition.

17. Creates anti aging, beauty wrinkle repair products with sulfur content. Hydro carbon protective skin creams with n butane and cis ash mine. Also gels, resins, saps, tars, botanicals, gums, proteins, distillates, juices, inositol emollients.

18. Creates flavor enhancers, they alter the response of the sensorary nerve endings on the tongue and in the nose.

19. Promotes healing, being neuroactive, neurochemistry and neuroscience.
